(19) United States
(12) Patent Application Publication
(10) Pub. No.: US 2015/0059776 A1
(43) Pub. Date: Mar. 5, 2015

(54) Earplug

(71) Applicant: Hung-Wen Hung, New Taipei City (TW)

(72) Inventor: Hung-Wen Hung, New Taipei City (TW)

(21) Appl. No.: 14/447,618

(22) Filed: Jul. 31, 2014

(30) Foreign Application Priority Data
Sep. 4, 2013 (TW) 102131752
Sep. 4, 2013 (TW) TW102216612

(51) Int. Cl. A61F 11/08

(52) U.S. Cl. CPC A61F 11/08; A61F 2011/085
USPC 128/867

(57) ABSTRACT

An earplug is provided to prevent the tinnitus, which comprises an earplug body and at least one tube. The earplug body is made from a soft moldable material. The tube is placed inside the earplug body surrounded by the soft moldable material, and being formed in a continuous curve shape that has two openings at two ends of the tube, and a diameter of the tube is in a range from 0.03 cm to 0.15 cm. The tube is configured for communicating outside air to maintain the balance of the air pressure, and thus the earplug of the present invention is able to prevent the tinnitus of the user. Further, although the earplug of the present invention blocks most of the external sound, it still allows some thimbleful sound entering through the tube. In this way, the user does not need to worry about losing contact with the outside and remain the ability of noise reduction, so the user may feel more relieved while sleeping.

### FIELD OF THE INVENTION

The present invention relates to an earplug, and particularly to an earplug for noise reduction.

### BACKGROUND

An earplug is used to protect human's ear from loud noise, it can block external sounds which may enter the internal acoustic meatus. Although the earplugs may have many different appearances, all the earplugs are made with the soft filler and modeled to a shape to stuff into the ear canal easily. When the user using the earplugs, the user can squeeze the earplug slightly and insert the earplug into the ear canal. Thus, the earplugs are produced by the soft filler which has the characteristics in good elastic and good plasticity to insert into the ear canal tightly.

However, when the user inserts the earplug into the ear canal, the earplug may changes the comfortable environment of the ear because of the ear canal has been block completely. It may induce the tinnitus to the user, and let the user feel uncomfortable. Moreover, if the user inserted the earplug into the ear canal, the user may worry about that he/she can not perceive the emergencies immediately, because the earplug blocks external sounds completely, and let the user have no peace of mind to sleep. It may affect the quality of sleeping to the user.

### SOME EXEMPLARY EMBODIMENTS

The object of this invention is to prevent the tinnitus of a user and let the user still can perceive if something is happening around himself/herself when the user is using an earplug.

In one embodiment of the present invention, an earplug comprises an earplug body and at least one tube. The earplug body is made from a soft moldable material, and has a first end and a second end. The tube is placed inside the earplug body surrounded by the soft moldable material, and is formed in a continuous curve with a very small diameter, each tube has a first opening and a second opening, wherein the first opening is placed to a surface of the first end of the earplug body and the second opening is placed to a surface of the second end of the earplug body. The tube is configured for communicating outside air to maintain the balance of the air pressure

Accordingly, through the tube which is placed in the earplug body, the present invention can prevent the tinnitus of the user by the external air to balance the air pressure in the ear canal. Furthermore, although the earplug of the present invention blocked most of the external sounds which may enter the ear canal, the earplug of the present invention still allows a thimbleful of the external sounds enter the ear canal through the tube, thus, the user does not need to worry about he/she may lost contact with the outside, so the user may feel more relieved when he/she is sleeping.

### BRIEF DESCRIPTION OF THE DRAWINGS

The above objects and advantages of the present invention will become more readily apparent to those ordinarily skilled in the art after reviewing the following detailed description and accompanying drawings, in which:

FIG. 1 is a sectional view of an earplug; and

FIG. 2 is another sectional view of an earplug.

### DESCRIPTION OF THE PREFERRED EMBODIMENT

The embodiments of the structure and/or methods are disclosed. In the following description, for purposes of explanation, numerous specific details are set forth in order to provide a thorough understanding of the embodiments of the disclosure. It is apparent, however, to one skilled in the art that the present disclosure may be practiced without these specific details or with an equivalent arrangement.

The embodiments of the present disclosure are able to apply in an earplug which is used to be inserted in the ear canal to protect the user's ears from loud noises.

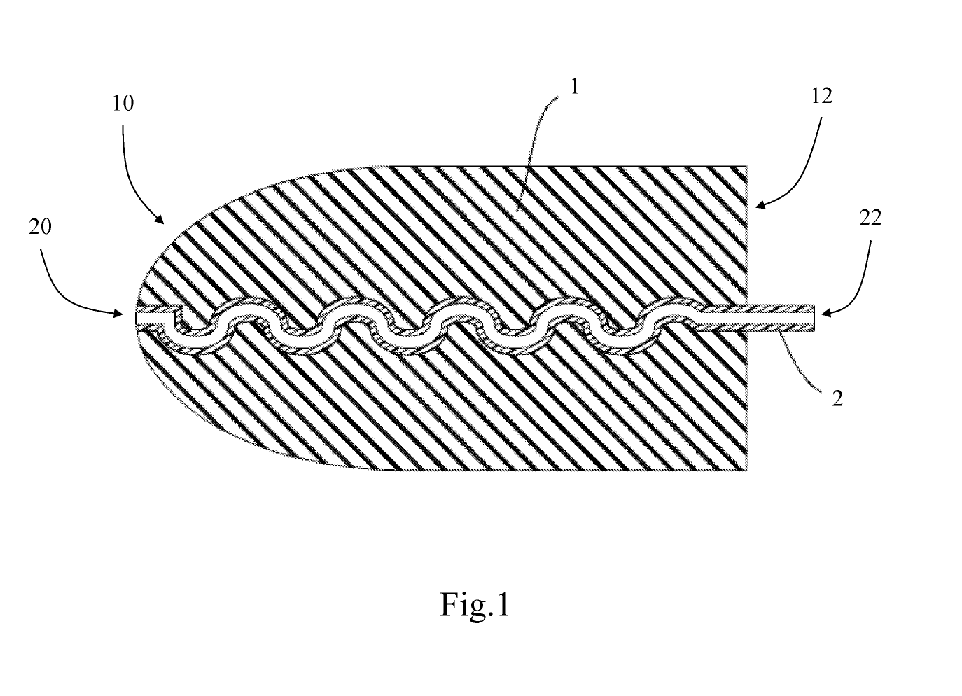

Please refer to FIG. 1. FIG. 1 is a sectional view of an earplug. The earplug comprises an earplug body 1 and at least one tube 2. The earplug body 1 is made from a soft moldable material (ex: a Silicone or a Silicone Putty), and the earplug body 1 has a first end 10 and a second end 12, wherein the first end 10 is a portion of the earplug body 1 which may be inserted to an ear canal, and the second end 12 is the rest of the earplug body 1 which is exposed to the outside of the ear canal when the user using the earplug.

The tube 2 has a very small diameter in a range from 0.03 centimeter (cm) to 0.15 centimeter (cm), and is placed inside the earplug body 1 surrounded by the soft moldable material. The tube 2 has a first opening 20 and a second opening 22, wherein the first opening 20 is placed to a surface of the first end 10 and the second opening 22 is placed to a surface of the second end 12, so the tube 2 can extend from the first end 10 to the second end 12, and let the first end 10 connected to the second end 12. Therefore, when the user using the earplug of the present invention, the earplug may allows a thimbleful of the external air passes through the earplug body 1 by the tube 2 to enter the ear canal.

Further, the cross sectional view of the tube 2 is in a circle, an oval, a triangle, a rectangle, or a rhombic shape, and the two openings 20, 22 of the tube 2 are selectively formed at the surface of the earplug body 1. In other words, the first opening 20 of the tube 2 may aligned with the surface of the first end 10 of the earplug body 1 and the second opening 22 of the tube 2 may aligned with the surface of the second end 12 of the earplug body 1.

In another embodiment, the tube 2 is protruded outwardly from the earplug body 1 which makes either one of the two openings 20, 22 outside of the earplug body 1 or both. In other words, the first opening 20 of the tube 2 may protruded outwardly from the first end 10 of the earplug body 1 and the second opening 22 of the tube 2 may protruded outwardly from the second end 12 of the earplug body 1, or the first opening 20 of the tube 2 is protruded outwardly from the first end 10 of the earplug body 1 but the second opening 22 of the tube 2 is aligned with the surface of the second end 12 of the earplug body 1, or the first opening 20 of the tube 2 is aligned with the surface of the first end 10 of the earplug body 1 but the second opening 22 of the tube 2 is protruded outwardly from the second end 12 of the earplug body 1.

Furthermore, the first opening 20 of the tube 2 and/or the second opening 22 of the tube 2 are/is formed in a trumpet-shaped. As the result, the diameter of the first opening 20 is bigger than the diameter of the second opening 22, or the diameter of the first opening 20 is smaller than the diameter of the second opening 22, or the diameter of the first opening 20 is equal to the diameter of the second opening 22.

In order to eliminate more external sounds, the tube 2 in an embodiment is formed in a continuous curve. In this case, when the external sounds cross through the tube 2 from the first opening 20 to the second opening 22, the external sound will be dissipated because the sound wave of the external sound may bumped into the turning point of the continuous curve of the tube 2. Thus, the user will almost hear the external sounds, but still can perceive if something is happening around himself/herself.

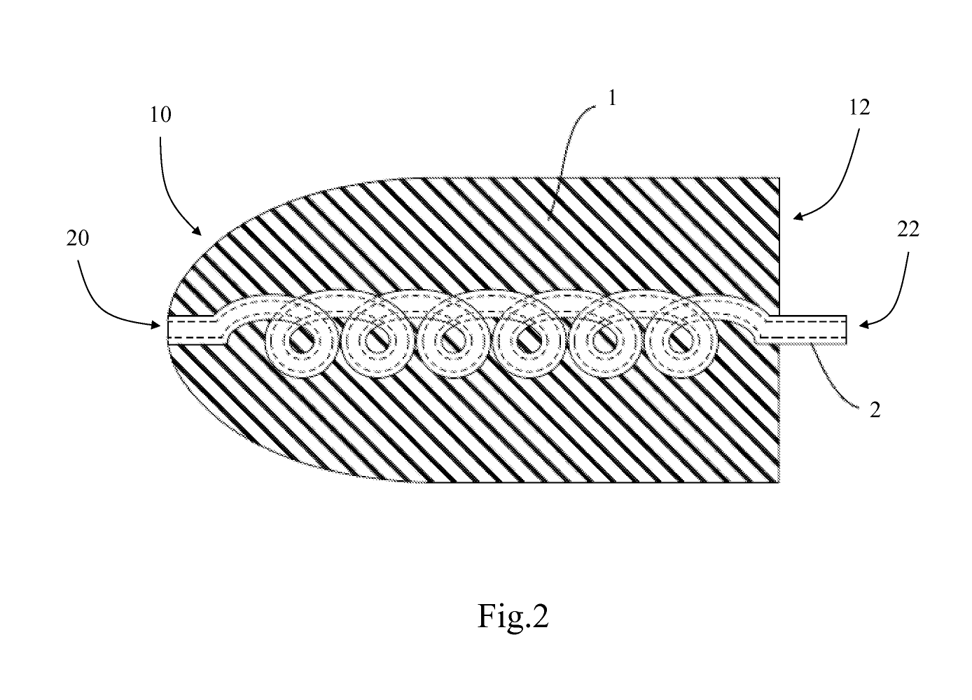

In addition, FIG. 2 shows another sectional view of an earplug. In this embodiment of the present invention, the tube 2 of the earplug is formed in a spiral. In this case, the external sound will be more difficult to cross through the tube 2 from the first opening 20 to the second opening 22.

Therefore, through the tube 2 which is placed in the earplug body 1, the earplug of the present invention can prevent the tinnitus of the user by the external air to balance the air pressure in the ear canal. Furthermore, although the earplug of the present invention blocks most of the external sounds (i.e., external environment sound or noise) which may enter the ear canal, the earplug of the present invention still allows a thimbleful of the external sounds enter the ear canal through the tube 2, thus, the user do not need to worry about he/she may lost contact with the outside, so the user may feel more relieved when he/she is sleeping.

What is claimed is:

1. An earplug, comprising:
an earplug body being made from a soft moldable material; and
at least one tube being placed inside the earplug body surrounded by the soft moldable material, and being formed in a continuous curve shape that has two openings at two ends of the tube, wherein the tube is configured for communicating outside air to maintain the balance of the air pressure, and allows environment thimbleful sound entering through thereof, and a diameter of the tube is in a range from 0.03 cm to 0.15 cm.

2. The earplug according to claim 1, wherein each two openings of the tube are formed at the surface of the earplug body.

3. The earplug according to claim 1, wherein the tube is protruded outwardly from the earplug body which makes either one of the two openings outside of the earplug body, or both.

4. The earplug according to claim 1, wherein either one of the two openings of the tube is formed in a trumpet-shape, or both.

5. The earplug according to claim 1, wherein a diameter of one of the two openings is either bigger than another, or equal to another.

6. The earplug according to claim 1, wherein a cross sectional view of the tube is in a circle, an oval, a triangle, a rectangle, or a rhombic shape.

7. The earplug according to claim 1, wherein the tube is formed in a spiral shape.

8. An earplug, comprising:
an earplug body being made from a soft moldable material, and having a first end and a second end; and
at least one tube being placed inside the earplug body surrounded by the soft moldable material, and being formed in a continuous curve shape that has a first opening and a second opening, wherein a diameter of the tube is in a range from 0.03 cm to 0.15 cm, and the first opening is placed to a surface of the first end of the earplug body and the second opening is placed to a surface of the second end of the earplug body.

9. The earplug according to claim 8, wherein the first opening of the tube is aligned with the surface of the first end of the earplug body and the second opening of the tube is aligned with the surface of the second end of the earplug body.

10. The earplug according to claim 8, wherein the first opening of the tube is protruded outwardly from the first end of the earplug body and the second opening of the tube is protruded outwardly from the second end of the earplug body.

11. The earplug according to claim 8, wherein the first opening of the tube is aligned with the surface of the first end of the earplug body and the second opening of the tube is protruded outwardly from the second end of the earplug body, or the first opening of the tube is protruded outwardly from the first end of the earplug body and the second opening of the tube is aligned with the surface of the second end of the earplug body.

12. The earplug according to claim 8, wherein the first opening of the tube and the second opening of the tube are both formed in a trumpet-shaped.

13. The earplug according to claim 8, wherein the first opening of the tube or the second opening of the tube is formed in a trumpet-shaped.

14. The earplug according to claim 8, wherein a diameter of the first opening is bigger than a diameter of the second opening.

15. The earplug according to claim 8, wherein a diameter of the first opening is smaller than a diameter of the second opening.

16. The earplug according to claim 8, wherein a diameter of the first opening is equal to a diameter of the second opening.

17. The earplug according to claim 8, wherein a cross sectional view of the tube is in a circle, an oval, a triangle, a rectangle, or a rhombic shape.

18. The earplug according to claim 8, wherein the tube is formed in a spiral shape.
